(19) United States
(12) Patent Application Publication
(10) Pub. No.: US 2022/0249300 A1
(43) Pub. Date: Aug. 11, 2022

(54) ABSORBENT ARTICLES WITH BACKSHEET GRAPHICS

(71) Applicant: The Procter & Gamble Company, Cincinnati, OH (US)

(72) Inventors: Danielle SCHIANO, Cincinnati, OH (US); Keith ALVARADO, Franklin, OH (US)

(21) Appl. No.: 17/168,683

(22) Filed: Feb. 5, 2021

(51) Int. Cl. A61F 13/496; A61F 13/84

(52) U.S. Cl. CPC A61F 13/496; A61F 2013/8497; A61F 13/84

(57) ABSTRACT

An absorbent article having a front waist region with a front waist edge, a back waist region with a back waist edge, and a crotch region disposed between the front and back waist regions is disclosed. The absorbent article has a topsheet, a backsheet, and an absorbent core positioned between the topsheet and the backsheet and having a front end edge and a back end edge. The backsheet has a plurality of graphics. Graphics of a first portion of the plurality of graphics disposed on a portion of the backsheet between the back waist edge of the absorbent article and the back end edge of the absorbent core have an opacity less than graphics of a second portion of the plurality of graphics disposed on a portion of the backsheet between the back end edge of the absorbent core and the front waist edge of the absorbent article.

### FIELD

The present disclosure is directed to absorbent articles comprising backsheets having a plurality of graphics, wherein graphics of a first portion of the plurality of graphics have a first opacity, and graphics of a second portion of the plurality of graphics have a second opacity.

### BACKGROUND

Absorbent articles are used in the hygiene industry to contain and absorb bodily exudates (i.e., urine and bowel movements) in infants, toddlers, children, and adults. Absorbent articles may include, but not be limited to, diapers and pants for infants and toddlers as well as adult incontinence pants. In some instances, a backsheet of an absorbent article may be embellished with graphics to appeal to the consumer, to make the absorbent article more like traditional underwear, and/or to communicate signals regarding proper placement and fit on a wearer. Diaper and pant-style absorbent articles typically have an absorbent core disposed at least partially between the topsheet and the backsheet, and functions to hold bodily exudates away from the skin of the wearer. Due to the placement of the absorbent core between the backsheet and the topsheet, the graphics disposed on the backsheet under the absorbent core are usually not visible from the wearer-facing side of the absorbent article. The absorbent core, however, does not typically overlap all of the backsheet. Rather, there is typically a portion of backsheet in the rear of the absorbent article that is not overlapped by the absorbent core and thus is visible from the wearer-facing side of the article, especially when the top sheet is of low basis weight. This portion of the rear of the absorbent article may be referred to as the “empty back” region. When the backsheet comprises graphics, the graphics may tend to be highly visible from the wearer-facing side of the absorbent article in the empty back region. This may be especially true when the backsheet does not comprise an underlay or base of color upon which the graphics are disposed. The use of underlays on backsheets, however can significantly increase the cost of the absorbent article. High visibility of backsheet graphics from the wearer-facing side may give consumers the impression that the article is thin, of low quality, and/or not rugged. As such, absorbent articles with backsheet graphics should be improved.

### SUMMARY

The present disclosure solves the problem discussed above by providing an absorbent article having a backsheet with a plurality of graphics. The graphics disposed on the portions backsheet not overlapped by the core may have less opacity, and the portion of the backsheet not overlapped by the core may have more graphic-free area, so that the graphics are less noticeable from a wearer-facing side of the absorbent article.

The present disclosure solves one or more of the problems discussed above by providing, in part, an absorbent article comprising a front waist region comprising a front waist edge, a back waist region comprising a back waist edge disposed opposite the front waist edge, and a crotch region disposed between the front and back waist regions. The absorbent article comprises a liquid permeable topsheet, a liquid impermeable backsheet, and an absorbent core positioned at least partially between the topsheet and the backsheet and comprising a front end edge disposed in the front waist region and a back end edge disposed in the back waist region. The backsheet comprises a plurality of graphics, wherein graphics of a first portion of the plurality of graphics disposed on a portion of the backsheet between the back waist edge of the absorbent article and the back end edge of the absorbent core have an opacity less than graphics of a second portion of the plurality of graphics disposed on a portion of the backsheet between the back end edge of the absorbent core and the front waist edge of the absorbent article, as measured according to the Opacity Test Method described herein.

The present disclosure provides, in part, an absorbent article comprising a front waist region comprising a front waist edge, a back waist region comprising a back waist edge disposed opposite the front waist edge, and a crotch region disposed between the front and back waist regions. The absorbent article comprises a liquid permeable topsheet, a liquid impermeable backsheet comprising a plurality of graphics, and an absorbent core positioned at least partially between the topsheet and the backsheet and comprising a front end edge disposed in the front waist region and a back end edge disposed in the back waist region. Graphics of a first portion of the plurality of graphics disposed on a garment-facing surface of the backsheet between the back waist edge of the absorbent article and the back end edge of the absorbent core have a first opacity, as measured according to the Opacity Test Method described herein. Graphics of a second portion of the plurality of graphics disposed on the garment-facing surface of the backsheet between the first portion of the graphics and the front waist edge of the absorbent article have a second opacity, as measured according to the Opacity Test Method described herein. The first opacity is different than the second opacity. A portion of the backsheet between the back waist edge of the absorbent article and the back end edge of the absorbent core may comprise more graphic-free area than the portion of the backsheet disposed between the back end edge of the absorbent core and the front waist edge of the absorbent article, as measured according to the Graphic-Free Area Test Method described herein.

The present disclosure provides, in part, an absorbent article comprising a front waist region comprising a front waist edge, a back waist region comprising a back waist edge disposed opposite the front waist edge, and a crotch region disposed between the front and back waist regions. The absorbent article comprises a liquid permeable topsheet, a liquid impermeable backsheet comprising a plurality of graphics, and an absorbent core positioned at least partially between the topsheet and the backsheet and comprising a front end edge disposed in the front waist region and a back end edge disposed in the back waist region. Graphics of a first portion of the plurality of graphics disposed on a garment-facing surface of the backsheet between the back waist edge of the absorbent article and the back end edge of the absorbent core have a first opacity, as measured according to the Opacity Test Method described herein. Graphics of a second portion of the plurality of graphics disposed on the garment-facing surface of the backsheet between the first portion of the graphics and the front waist edge of the absorbent article have a second opacity, as measured according to the Opacity Test Method described herein. The first opacity may be between about 5% and about 90% of the second opacity. A portion of the backsheet between the back waist edge of the absorbent article and the back end edge of the absorbent core may comprise between about 20% and about 80% more graphic-free area than a portion of the backsheet disposed between the back end edge of the absorbent core and the front waist edge of the absorbent article, as measured according to the Graphic-Free Area Test Method described herein.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 10;

FIG. 12 is a plan view of an example absorbent article in the form of a taped diaper with backsheet graphics, garment-facing surface facing the viewer, in a flat laid-out state;

FIG. 13 is a plan view of an example absorbent article in the form of a taped diaper with backsheet graphics and live leg zones, garment-facing surface facing the viewer, in a flat laid-out state;

FIG. 14 is a plan view of a backsheet comprising a plurality of graphics comprising a plurality of line segment groups;

FIG. 15 is a plan view of a backsheet comprising a plurality of graphics comprising a series of crossed line segments;

FIG. 16 is a plan view of an example absorbent article in the form of a pant with belt graphics, garment-facing surface facing the viewer, in a flat laid-out state; and

FIG. 17 is a plan view of an example absorbent article in the form of a pant with belt and chassis graphics, garment-facing surface facing the viewer, in a flat laid-out state.

### DETAILED DESCRIPTION

Various non-limiting forms of the present disclosure will now be described to provide an overall understanding of the principles of the structure, function, manufacture, and use of the absorbent articles comprising backsheet graphics disclosed herein. One or more examples of these non-limiting forms are illustrated in the accompanying drawings. Those of ordinary skill in the art will understand that the absorbent articles comprising backsheet graphics described herein and illustrated in the accompanying drawings are non-limiting example forms. The features illustrated or described in connection with one non-limiting form may be combined with the features of other non-limiting forms. Such modifications and variations are intended to be included within the scope of the present disclosure. Elements of the figures having the same reference number may be the same element.

### General Description of an Absorbent Article

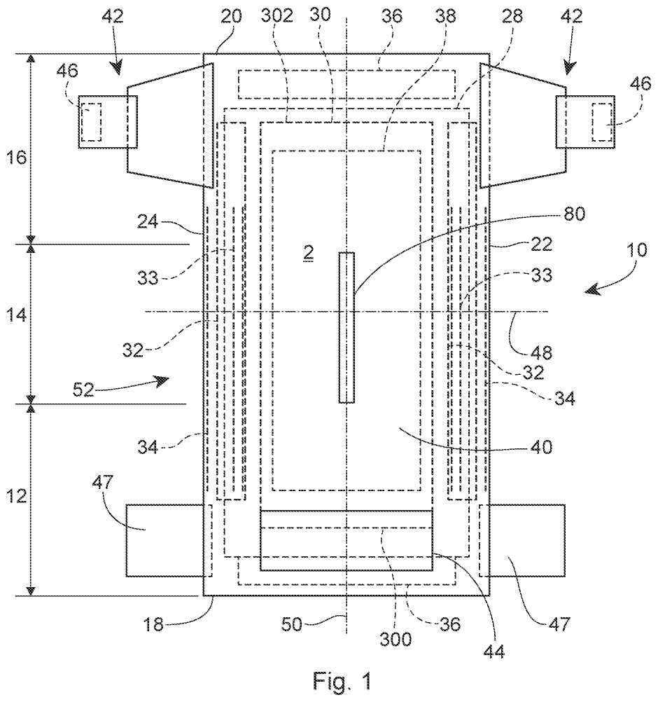

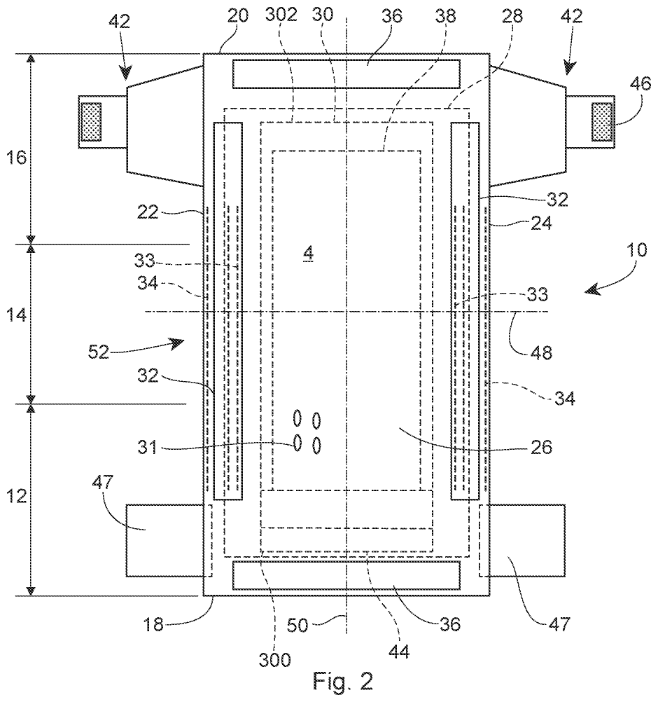

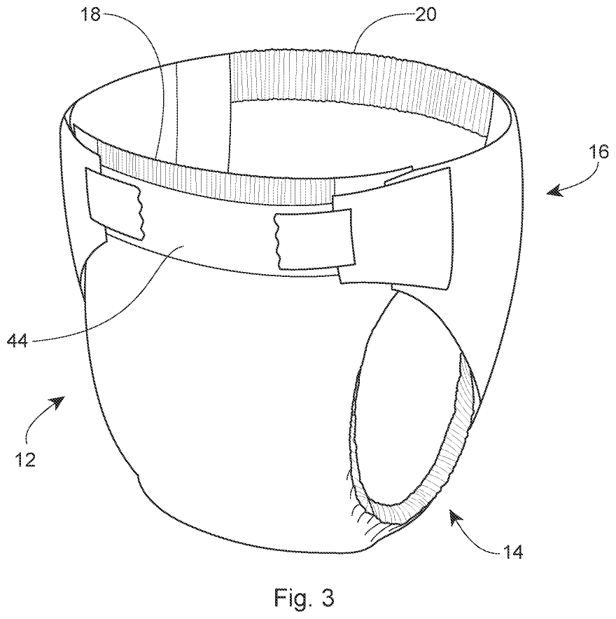

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of absorbent articles, including adult incontinence products and pants-style diapers, for example.

The absorbent article 10 may comprise a front waist region 12, a crotch region 14, and a back waist region 16. The crotch region 14 may extend between the front waist region 12 and the back waist region 16. The front wait region 12, the crotch region 14, and the back waist region 16 may each be about 1/3 of the length of the absorbent article 10. The absorbent article 10 may comprise a front waist edge 18, a back waist edge 20 opposite to the front waist edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially between the topsheet 26 and the backsheet 28. The absorbent core 30 may comprise a front end edge 300 disposed in the front waist region 18 and a back end edge 302 disposed in the back waist region 16. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned between the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

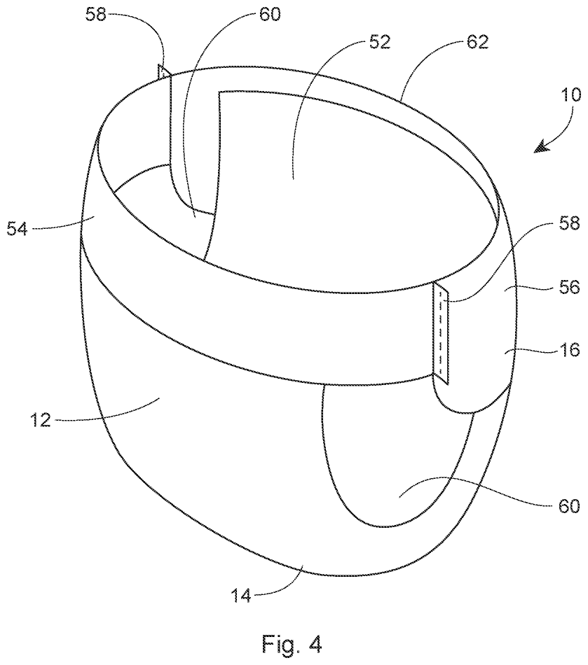

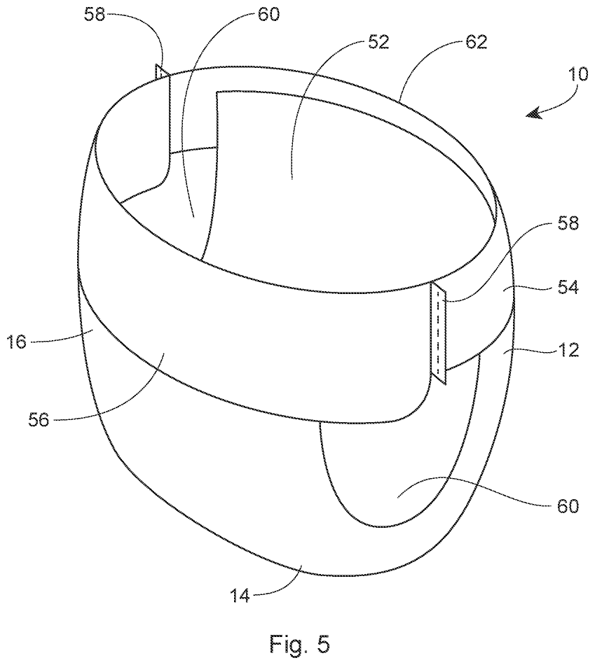

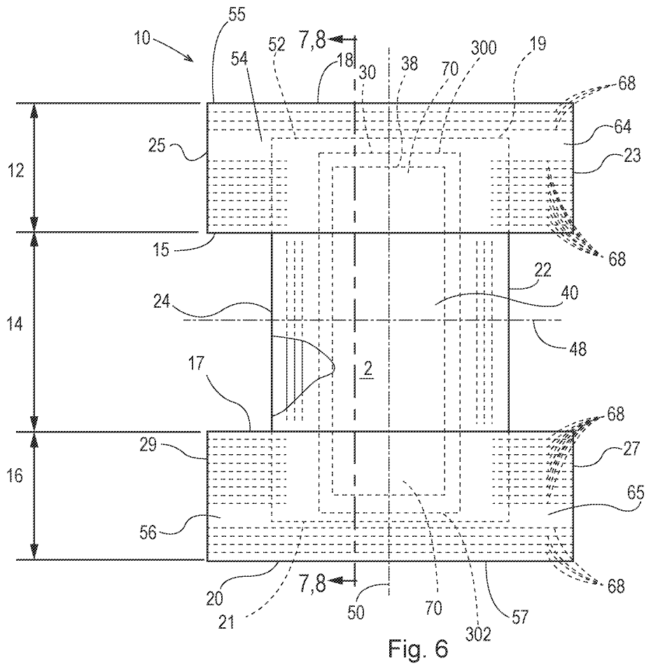

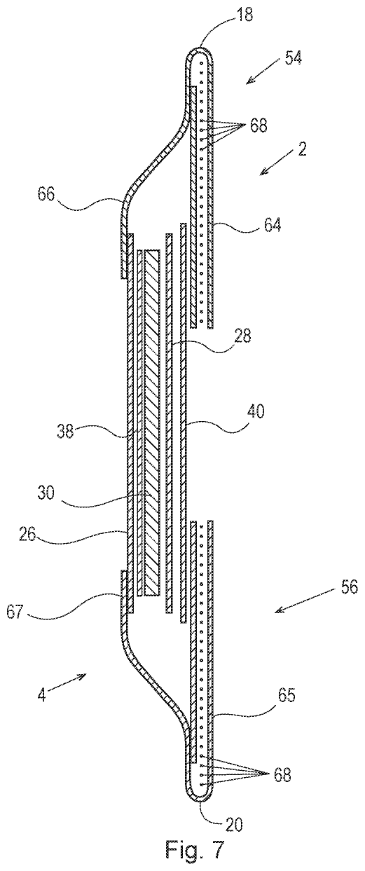

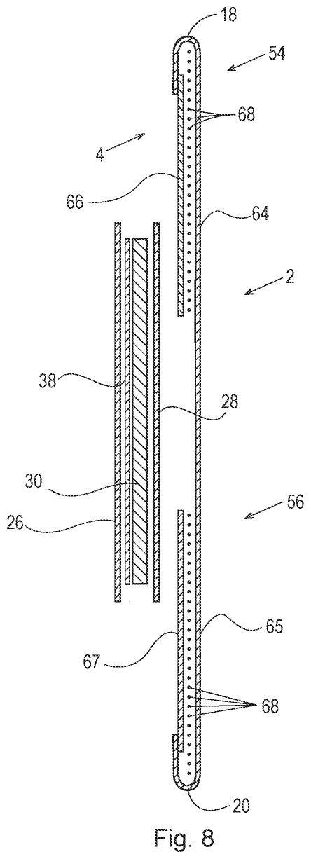

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially between the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. Appl. Pub. No. 2013/0211363.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material.

### Absorbent Core

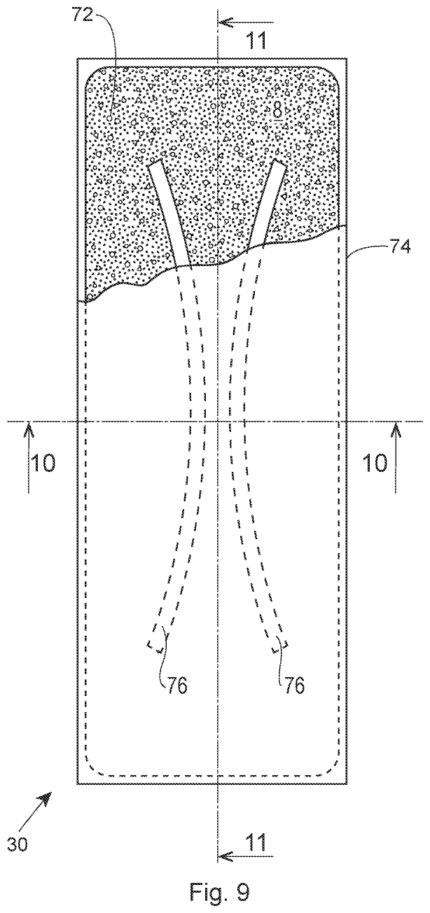

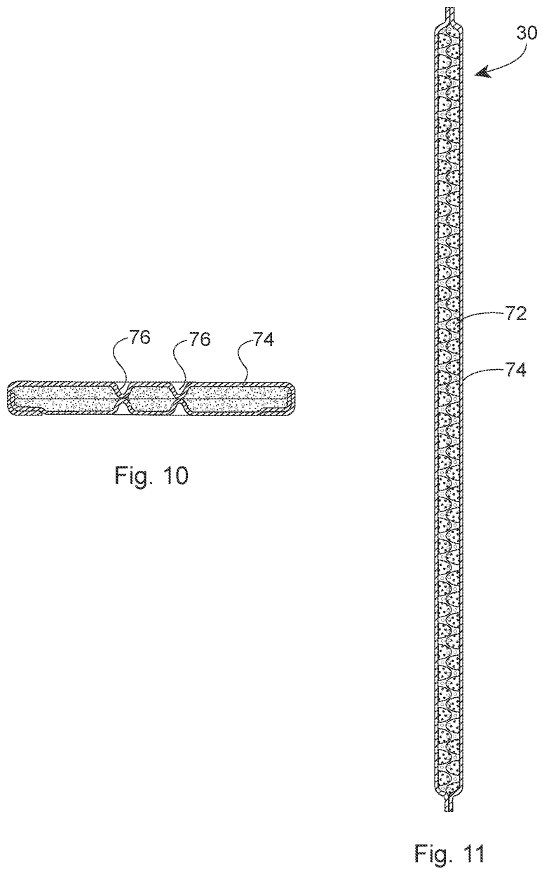

As used herein, the term “absorbent core” 30 refers to the component of the absorbent article 10 having the most absorbent capacity and that comprises an absorbent material. Referring to FIGS. 9-11, in some instances, absorbent material 72 may be positioned within a core bag or a core wrap 74. The absorbent material may be profiled or not profiled, depending on the specific absorbent article. The absorbent core 30 may comprise, consist essentially of, or consist of, a core wrap, absorbent material 72, and glue enclosed within the core wrap. The absorbent material may comprise superabsorbent polymers, a mixture of superabsorbent polymers and air felt, only air felt, and/or a high internal phase emulsion foam. In some instances, the absorbent material may comprise at least 80%, at least 85%, at least 90%, at least 95%, at least 99%, or up to 100% superabsorbent polymers, by weight of the absorbent material. In such instances, the absorbent material may be free of air felt, or at least mostly free of air felt. The absorbent core periphery, which may be the periphery of the core wrap, may define any suitable shape, such as rectangular “T,” “Y,” “hour-glass,” or “dog-bone” shaped, for example. An absorbent core periphery having a generally “dog bone” or “hour-glass” shape may taper along its width towards the crotch region 14 of the absorbent article 10.

Referring to FIGS. 9-11, the absorbent core 30 may have areas having little or no absorbent material 72, where a wearer-facing surface of the core bag 74 may be joined to a garment-facing surface of the core bag 74. These areas having little or no absorbent material and may be referred to as “channels” 76. These channels can embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front waist edge 18 and the back waist edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front waist edge 18 and the back waist edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. As an example, a first elastic waistband 36 may be present in the front waist region 12 near the front belt waist edge 18 and a second elastic waistband 36 may be present in the back waist region 16 near the back waist edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 75 and the liquid impermeable backsheet 28.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g. color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate number of absorbent articles per package. By packaging the absorbent articles under compression, caregivers can easily handle and store the packages, while also providing distribution savings to manufacturers owing to the size of the packages.

### Arrays

“Array” means a display of packages comprising disposable absorbent articles of different article constructions (e.g., different elastomeric materials [compositionally and/or structurally] in the side panels, side flaps and/or belts flaps, different graphic elements, different product structures, fasteners or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example,

“Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Bio-Based Content for Components

Components of the absorbent articles described herein may at least partially be comprised of bio-based content as described in U.S. Pat. Appl. No. 2007/0219521 A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S. A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

### Backsheet Graphics

Absorbent articles of the present disclosure may comprise backsheets comprising a plurality of graphics. As used herein, the term “graphic” or “graphics” refers to a colored area of the backsheet. Color may be realized on the backsheet by dying the backsheet or a portion thereof, by applying an ink on the surface of the backsheet, for example, by printing on a surface of the backsheet, by incorporating a pigment into the backsheet or portions thereof, or by any other method of applying a color known in the art. A “graphic” may include the depiction of a design or designs, any recognizable indicia, such as a number, a letter, a word, a brand name, an icon, a logo, a character, a portion of, or all of, a wetness indicator, a front/back indicator, any shape and/or symbol (for example hearts, clouds, animals, etc.), as well as a full flood of pigment across a surface, or a portion of a surface, of the backsheet. “Graphics” may also comprise visible placement indicia to indicate where a sensor should be attached to the absorbent article, such as a dashed outline that matches the shape of a sensor, for example.

The plurality of graphics are functionally related to the backsheet they are disposed on because, aside from adding interest and design elements to the garment-facing surface of the absorbent article, guiding proper fit of the absorbent article, and/or guiding placement of sensors, portions of the plurality of graphics may be visible through the absorbent article to the wearer-facing surface. High visibility of backsheet graphics from the wearer-facing side may give consumers the impression that the article is thin, of low quality, and/or not rugged. Thus, the plurality of graphics may serve as an indicator of absorbent article quality and robustness.

The plurality of graphics of the backsheet may comprise any one color or color tone, or any combination of colors and/or color tones. The colors and/or color tones may be characterized by L*, a*, b* values, according to the CIE L*a*b* Test Method described herein. The CIE L*, a*, b* color scale utilizes measures of lightness (L*), redness-greenness (a*), and yellowness-blueness (b*) to characterize colors. The plurality of graphics of the backsheet may have only one color tone.

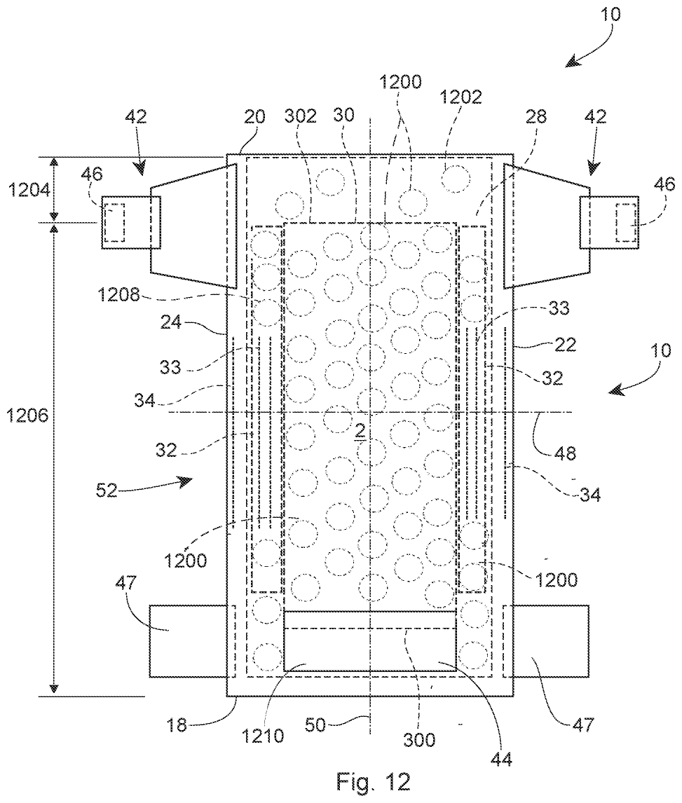

The plurality of graphics may be positioned on a wearer-facing surface and/or a garment-facing surface of the backsheet. The plurality of graphics may be arranged such that a first portion of the graphics are disposed on a first portion of the backsheet, and a second portion of the graphics are disposed on a second portion of the backsheet. Referring to FIG. 12, a plurality of graphics 1200 may be disposed on at least a portion of the backsheet 28, such that a first portion of the graphics 1202 is disposed on a first portion of the backsheet 1204 between a back waist edge 20 of the absorbent article 10 and a back end edge 302 of the absorbent core 30. A second portion of the graphics 1208 may be disposed on a second portion of the backsheet 1206 between the back end edge 302 of the absorbent core 30 and a front waist edge 18 of the absorbent article 10.

The graphics 1200 forming the first portion of the graphics 1202 may have an opacity less than the graphics 1200 forming the second portion of the graphics 1208, according to the Opacity Test Method described herein. The graphics 1200 forming the first portion of the graphics 1202 may have an opacity of between about 5% and about 90%, between about 10% and about 85%, between about 15% and about 80%, or between about 20% and about 75% of the opacity of the graphics 1200 forming the second portion of the graphics 1208, specifically reciting every 1.0% increment within these ranges and any ranges formed therein or thereby, according to the Opacity Test Method described herein. Placement of graphics 1200 with less opacity on the first portion of the backsheet 1204—where there is no absorbent core 30 between the backsheet 28 and the wearer-facing surface 4 of the absorbent article 10—may reduce the visibility of the graphics 1200 in the first portion of the backsheet 1204 when the absorbent article 10 is viewed from the wearer-facing surface 4 of the absorbent article 10. This may have the benefit of giving the absorbent article a more homogeneous appearance when viewed from the wearer-facing surface, which may give the impression of a thicker, higher quality, and more rugged absorbent article. All of the graphics forming the first portion of the graphics 1202 may have an opacity less than all of the graphics 1200 forming the second portion of the graphics 1208, according to the Opacity Test Method described herein.

The graphics 1200 forming the first portion of the graphics 1202 may have an L* value greater than an L* value of the graphics 1200 forming the second portion of the graphics 1208, according to the CIE L*a*b* Test Method described herein. The graphics 1200 forming the first portion of the graphics 1202 may have an L* value of between about 10 and about 75, between about 15 and about 55, between about 20 and about 50, or between about 25 and about 45, specifically reciting each 1.0 increment within these ranges and any ranges formed therein or thereby, according to the CIE L*a*b* Test Method described herein. The graphics 1200 forming the second portion of the graphics 1208 may have an L* value of between about 0 and about 50, between about 0 and about 40, between about 0 and about 30, or between about 0 and about 20, specifically reciting each 1.0 increment within these ranges and any ranges formed therein or thereby, according to the CIE L*a*b* Test Method described herein. Because L* value represents a scale of blackness and whiteness (0=black and 100=white), the L* value of a graphic may correlate to opacity, as graphics having lower L* values (more black) may be more opaque as compared to graphics having higher L* values (more white). For example, when a backsheet comprises a white substrate, a graphic with a higher L* value (whiter) may be generated by using less colorant as opposed to using a colorant containing more white pigment. Thus, the graphic may both have a higher L* value and may be less opaque. All of the graphics 1200 forming the first portion of the graphics 1202 may have an L* value greater than an L* value of all of the graphics 1200 forming the second portion of the graphics 1208, according to the CIE L*a*b* Test Method described herein.

Still referring to FIG. 12, the first portion of the backsheet 1204 between the back waist edge 20 of the absorbent article 10 and the back end edge 302 of the absorbent core 30 may comprise more graphic-free area than the second portion of the backsheet 1206 between the back end edge 302 of the absorbent core and the front waist edge 18 of the absorbent article 10, as measured according to the Graphic-Free Area Test Method described herein. Having more graphic-free area on the first portion of the backsheet 1204 may reduce the appearance of the graphics 1200 in the first portion of the backsheet 1204 when the absorbent article 10 is viewed from the wearer-facing surface 4 of the absorbent article 10. This may have the benefit of giving the absorbent article a more homogeneous appearance when viewed from the wearer-facing surface, which may give the impression of a thicker, higher quality, and more rugged absorbent article. The first portion of the backsheet 1204 may comprises between about 20% and about 80%, between about 25% and about 75%, or between about 30% and about 60% more graphic-free area than the second portion of the backsheet 1206 , specifically reciting every 1.0% increment within these ranges and any ranges formed therein or thereby, as measured according to the Graphic-Free Area Test Method described herein.

The backsheet 28 may be free of an underlay. As used herein, the term “underlay” refers to a base of color applied to a surface—for example a surface of a backsheet—upon which graphics may be disposed. An underlay may comprise a single color or color tone, or may comprise more than one color and/or color tone. Typically, an underlay may provide a generally consistent background so as not to compete with graphics disposed thereon. An underlay may be a flood of color covering an entire side of the backsheet, or an underlay may be a discreet area of color disposed on the backsheet that is larger than a graphic disposed thereon. An underlay may function to prevent, or at least partially inhibit, a graphic from showing through to an opposite side of a garment. For example, where a graphic is disposed on a garment-facing surface of an absorbent article, an underlay may prevent, or at least partially inhibit, the graphic from being seen from the wearer-facing side of the absorbent article. This may give a consumer the impression that the absorbent article is thicker, of higher quality, and/or more rugged then when a graphic is visible through the absorbent article. The use of underlay, however, may significantly increase the cost of an absorbent article due to the increased use of ink needed to form the underlay. Absorbent articles of the present disclosure, comprising a first portion of graphics disposed between the back waist edge of the absorbent article and the back end edge of the absorbent core, and a second portion of graphics disposed between the back end edge of the absorbent core and the front waist edge, wherein the first portion of the graphics has an opacity less than the second portion of the graphics, may be free of an underlay while still maintaining the qualities of appearing thicker, higher quality, and more rugged. Therefore, absorbent articles of the present disclosure may be more cost effective to produce as compared to an absorbent article comprising an underlay.

Referring still to FIG. 12, the absorbent article 10 may comprise a backsheet 28 comprising a landing zone region 1210 that overlaps the landing zone area 44 of the outer cover material 40. The landing zone region 1210 of the backsheet 28 may be generally the same size and/or the same shape as the landing zone area 44 of the outer cover material 40. The landing zone region 1210 of the backsheet 28 may be between about 30 cm² and about 150 cm², between about 45 cm² and about 125 cm², or between about 50 cm² and about 110 cm², specifically reciting every 1 cm² increment within these ranges and any ranges formed therein or thereby. The landing zone region 1210 of the backsheet 28 may have more graphic-free area than a portion of the backsheet 28 between the back end edge 302 of the absorbent core 30 and the front wait edge 18 of the absorbent article 10, as measure according to the Graphic-Free Area Test Method described herein.

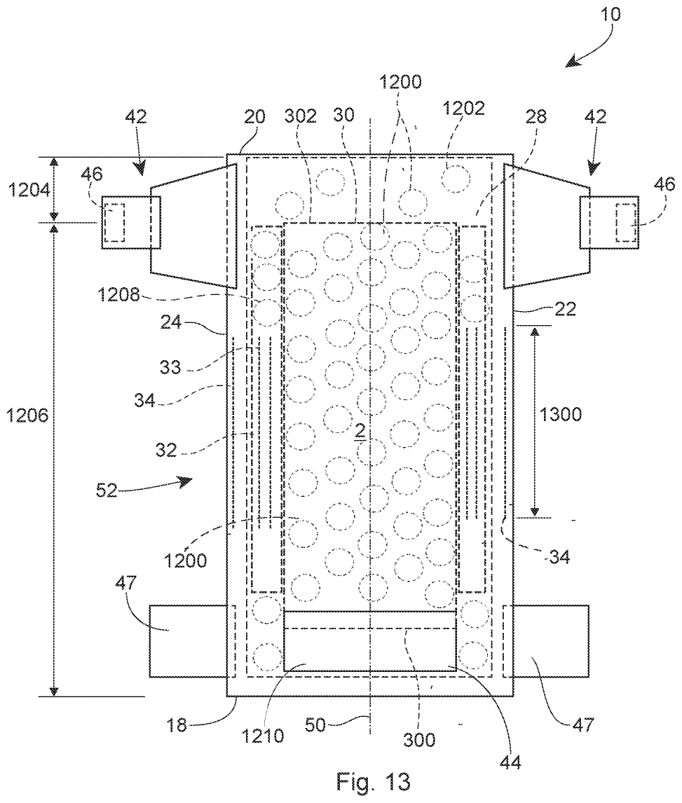

Referring to FIG. 13, an absorbent article 10 of the present disclosure may comprise a live leg zone 1300. As used herein, the term “live leg zone” refers to a portion of the absorbent article proximate at least one of the side edges 22 and/or 24 which comprise leg elastics 34 bonded to a portion of the absorbent article under tension, resulting in the formation of gathers at the side edges 22 and/or 24 when the absorbent article is in a relaxed state. At least a portion of the live leg zone 1300 may be free of graphics. Because the live leg zone comprises gathers that may disrupt the visual continuity of graphics disposed on the backsheet, an absorbent article of the present disclosure comprising a live leg zone, wherein at least a portion of the live leg zone is free of graphics, may provide an absorbent article with a cleaner and neater appearance, which may be consumer desired.

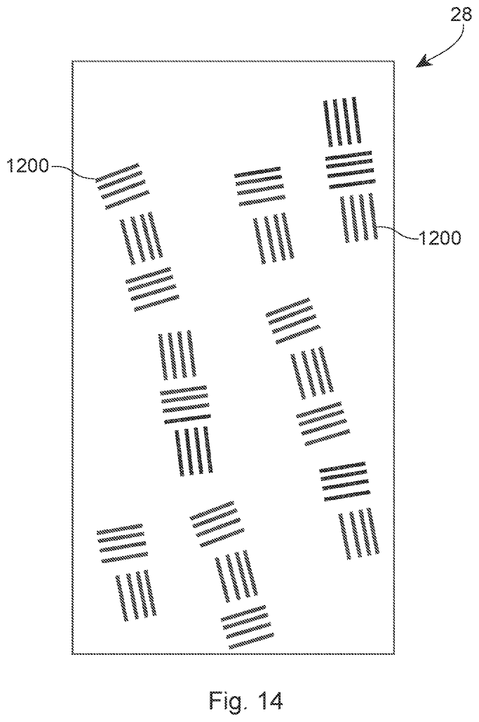

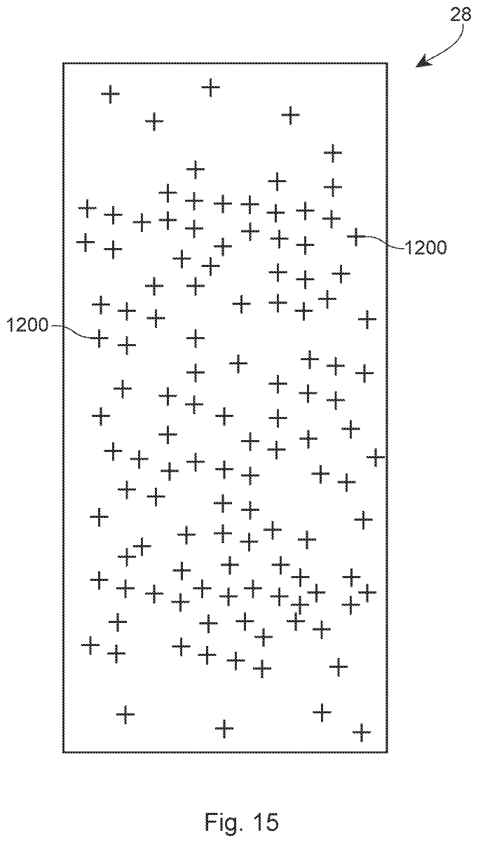

The plurality of graphics may be of any size and/or shape. As shown in FIG. 13, the plurality of graphics 1200 disposed on the backsheet 28 may comprise generally circular dots. As shown in FIG. 14, the plurality of graphics 1200 disposed on the backsheet 28 may comprise a plurality of line segment groups, wherein lines of each of the plurality of line segment groups are arranged generally parallel to one another, and wherein each plurality of line segment groups generally forms the shape of a square or rectangle. As shown in FIG. 15, the plurality of graphics 1200 disposed on the backsheet 28 may comprise a plurality of crossed line segments.

The plurality of graphics may comprise a wetness indicator. Referring to FIG. 1, wetness indicators 80 may be applied to the absorbent core facing side of the backsheet 28, so that they may be contacted by bodily exudates within the absorbent core. In some instances, the wetness indicators 80 may form portions of the plurality of graphics 1200. The wetness indicators 80 may appear or disappear and create or remove portions of some of the plurality of graphics 1200. In other instances, the wetness indicators 80 may coordinate (e.g., same design, same pattern, same color) or not coordinate with the plurality of graphics 1200.

The plurality of graphics may have a* values between 15 and −15, and b* values between 10 and −10, specifically reciting each value within these ranges and any ranges formed therein or thereby, according to the CIE L*a*b* Test Method described herein. a* and b* values near 0 may provide graphics that appear black or white, with little to no red, green, blue, or yellow appearance.

### Belt Graphics

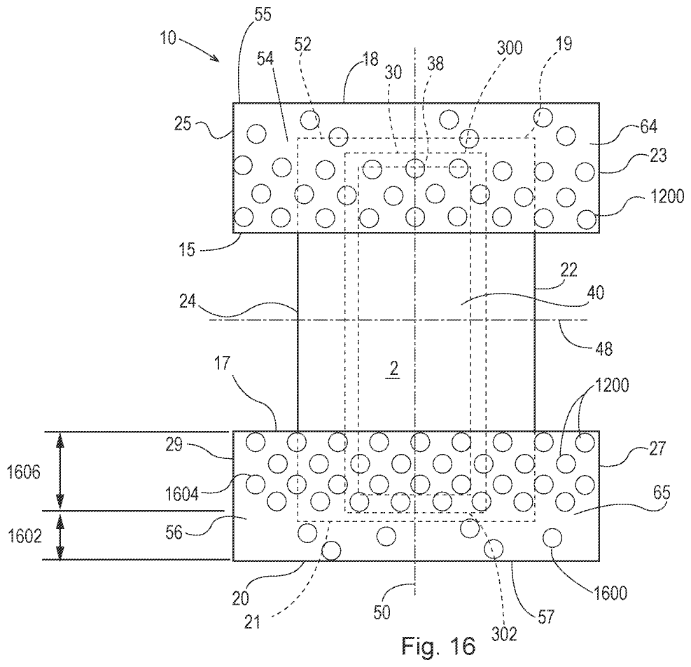

Referring to FIG. 16, the front and back belts 54 and 56 may comprise a plurality of graphics 1200. The plurality of graphics 1200 may be disposed on a garment-facing surface of the front and/or back belts 54 and 56, or may be disposed on an underlying layer of the front and/or back belts 54 and 56, such as an elastic film, for example. The plurality of graphics 1200 may be visible from a garment-facing side of the front and/or back belts 54 and 56. The plurality of graphics 1200 may be disposed on at least a portion of the back belt 56, such that a first portion of the graphics 1600 is disposed on a first portion 1602 of the back belt 56 between the back waist edge 20 of the absorbent article 10 and the back end edge 302 of the absorbent core 30. A second portion of the graphics 1604 may be disposed on a second portion 1606 of the back belt 56 between the back end edge 302 of the absorbent core 30 and the proximal back belt seam 17.

The graphics 1200 forming the first portion of the graphics 1600 may have an opacity less than the graphics 1200 forming the second portion of the graphics 1604, according to the Opacity Test Method described herein. The graphics 1200 forming the first portion of the graphics 1600 may have an opacity of between about 5% and about 90%, between about 10% and about 85%, between about 15% and about 80%, or between about 20% and about 75% of the opacity of the graphics 1200 forming the second portion of the graphics 1604, specifically reciting every 1.0% increment within these ranges and any ranges formed therein or thereby according to the Opacity Test Method described herein. Placement of graphics 1200 with less opacity on the first portion 1602 of the back belt 56—where there is no absorbent core 30 between the back belt 56 and the wearer-facing surface 4 of the absorbent article—may reduce the visibility of the graphics 1200 in the first portion of the back belt 56 when the absorbent article 10 is viewed from the wearer-facing surface 4 of the absorbent article 10. This may have the benefit of giving the absorbent article a more homogeneous appearance when viewed from the wearer-facing surface, which may give the impression of a thicker, higher quality and more rugged absorbent article.

The graphics 1200 forming the first portion of the graphics 1600 may have an L* value greater than an L* value of the graphics 1200 forming the second portion of the graphics 1604, according to the CIE L*a*b* Test Method described herein. The graphics 1200 forming the first portion of the graphics 1600 may have an L* value of between about 10 and about 75, between about 15 and about 55, between about 20 and about 50, or between about 25 and about 456, specifically reciting each 1.0 increment within these ranges and any ranges formed therein or thereby, according to the CIE L*a*b* Test Method described herein. The graphics 1200 forming the second portion of the graphics 1604 may have an L* value of between about 0 and about 50, between about 0 and about 40, between about 0 and about 30, or between about 0 and about 20, specifically reciting each 1.0 increment within these ranges and any ranges formed therein or thereby, according to the CIE L*a*b* Test Method described herein. Because L* value represents a scale of blackness and whiteness (0=black and 100=white), the L* value of a graphic may correlate to opacity, as graphics having lower L* values (more black) may be more opaque as compared to graphics having higher L* values (more white). For example, when a backsheet comprises a white substrate, a graphic with a higher L* value (whiter) may be generated by utilizing less colorant as opposed to a colorant containing more white pigment. Thus, the graphic may both have a higher L* value and may be less opaque.

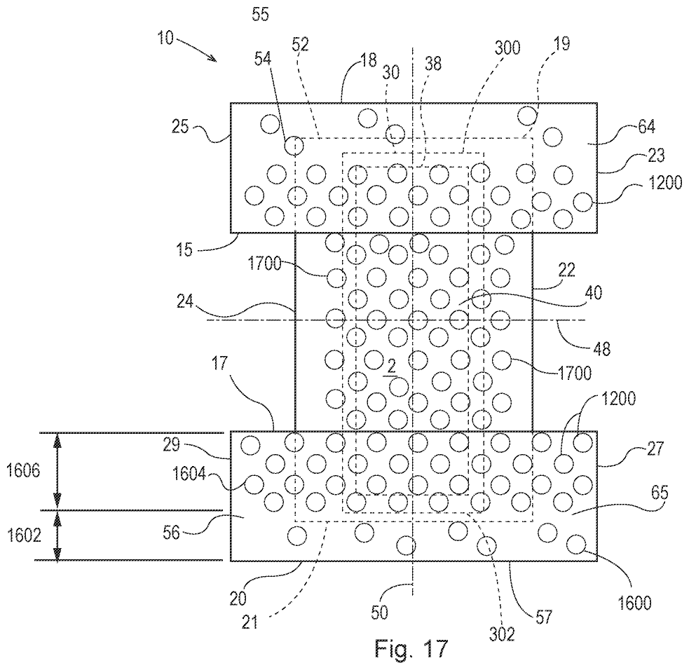

Referring to FIG. 17, the plurality of graphics 1200 disposed on the front and/or back belts 54 and 56 may appear similar to, coordinate with, or be different than graphics disposed on the chassis 1700. The graphics disposed on the chassis 1700 may have a generally similar opacity and/or L* value as the graphics 1200 forming the second portion of the graphics 1604 on the front and/or back belts 54 and 56. Where the plurality of graphics 1200 disposed on the front and/or back belts 54 and 56 have a similar or coordinated appearance as the graphics disposed on the chassis 1700, the absorbent article may have a unified and neat appearance, which may be consumer preferred.

### Test Methods

### Opacity Test Method:

Opacity by contrast ratio measurements are made using a 0°/45° spectrophotometer with adjustable apertures capable of making standard CIE color measurements using XYZ coordinates. An example of a suitable spectrophotometer is the Labscan XE (available from Hunter Associates Laboratory, Inc., Reston, Va., or equivalent). Measurements are conducted on a single layer of backsheet film test material. All testing is performed in a room maintained at a temperature of 23° C. ±2.0° C. and a relative humidity of 50% ±2% and samples are conditioned under the same environmental conditions for at least 2 hours prior to testing.

To obtain a sample, first secure an absorbent article to a rigid flat surface in a planar configuration with the garment-facing surface up. Next, mark the on the backsheet film layer the location of the back end edge of the absorbent core, such that the portion of the backsheet between the back waist edge of the absorbent article and back end edge of the absorbent core can be distinguished from the portion of the backsheet between the back end edge of the absorbent core and front waist edge of the absorbent article. Carefully remove the sample of backsheet film layer material from the absorbent article. A cryogenic spray (such as Cyto-Freeze, Control Company, Houston Tex.) can be used to remove the sample from the underlying and overlaying layers if necessary. Identify a graphic testing site on the sample located within the identified portion of the backsheet between the back waist edge of the absorbent article and back end edge of the absorbent core. If the sample at the testing site contains any holes, tears, or other physical deformations, another site is to be selected. Ensure that all adhesive and nonwoven fibers have been completely removed from the testing site. Obtain a sufficient quantity of the backsheet film layer sample material such that ten replicate measurements can be made on graphics located within the portion of the backsheet between the back waist edge of the absorbent article and back end edge of the absorbent core.

To measure Opacity, select the disk with the largest measurement port size that can fit within the selected graphic testing site. Calibrate and standardize the instrument per the vendor instructions using the standard white and black tiles provided by the vendor. Set the spectrophotometer to use the CIE XYZ color space with a D65 standard illumination, a 10° observer, and set the UV filter to nominal. Place the garment-facing surface of the test sample over the aperture and ensure that the entire aperture opening is covered by the testing site graphic. Place the standard white tile directly against the back side of the sample, take a reading and record the Y value as Y_{white backing} to the nearest 0.01 units. Without moving the position of the test sample, remove the standard white tile and replace it with the black standard tile. Take a reading and record the Y value as Y_{black backing} to the nearest 0.01 units. Calculate Opacity by dividing the Y_{black backing} value by the Y_{white backing} value and then multiply by 100. Record Opacity to the nearest 0.1 percent.

In like fashion, repeat the testing procedure for a total of ten measurements on graphics located within the portion of the backsheet between the back waist edge of the absorbent article and back end edge of the absorbent core. Calculate the arithmetic mean for Opacity obtained from all ten measurements and report to the nearest 0.1 percent.

Using the same prepared backsheet film samples, repeat the testing procedure on ten replicate graphics located within the portion of the backsheet between the back end edge of the absorbent core and front waist edge of the absorbent article. Calculate the arithmetic mean for Opacity obtained from all ten measurements and report to the nearest 0.1 percent.

### CIE L*a*b* Test Method:

Color analyses are made using a 0°/45° spectrophotometer with adjustable apertures capable of making standard CIE L*a*b* measurements in accordance with ASTM E1349. An example of a suitable spectrophotometer is the Labscan XE (available from Hunter Associates Laboratory, Inc., Reston, Va., or equivalent). All testing is performed in a room maintained at a temperature of 23° C. ±2.0° C. and a relative humidity of 50% ±2% and samples are conditioned under the same environmental conditions for at least 2 hours prior to testing.

To obtain a sample, first secure an absorbent article to a rigid flat surface in a planar configuration with the garment-facing surface up. Next, mark the on the backsheet film layer the location of the back end edge of the absorbent core, such that the portion of the backsheet between the back waist edge of the absorbent article and back end edge of the absorbent core can be distinguished from the portion of the backsheet between the back end edge of the absorbent core and front waist edge of the absorbent article. Carefully remove the sample of backsheet film layer material from the absorbent article. A cryogenic spray (such as Cyto-Freeze, Control Company, Houston Tex.) can be used to remove the sample from the underlying and overlaying layers if necessary. Identify a graphic testing site on the sample located within the identified portion of the backsheet between the back waist edge of the absorbent article and back end edge of the absorbent core. If the sample at the testing site contains any holes, tears, or other physical deformations, another site is to be selected. Ensure that all adhesive and nonwoven fibers have been completely removed from the testing site. Obtain a sufficient quantity of the backsheet film layer sample material such that ten replicate measurements can be made on graphics located within the portion of the backsheet between the back waist edge of the absorbent article and back end edge of the absorbent core.

Select the disk with the largest measurement port size that can fit within the selected homogeneous colored graphic testing site. Standardize the instrument using the selected port size (indicate the appropriate area view to the software) utilizing the manufacturer supplied black tile, then white tile. Calibrate the instrument according to manufacturer's specifications using their supplied standard tiles. Configure the software to measure color using the CIE 1976 L*a*b* color scale, D65 illuminant and 10° standard observer.

Place the sample over the measurement port, with the garment-facing surface oriented toward the instrument. Gently pull the sample taut, without stretching, to ensure that it does not pillow into the port, and then back it with the standard white tile. Make sure that the area of the sample to be measured faces the port and completely covers the port. Take a reading and record the individual L*, a*, and b* values, then remove the white tile and sample. The individual L*, a*, and b* values are recorded to the nearest 0.1 units.

In like fashion, repeat for a total of ten measurements on graphics located within the portion of the backsheet between the back waist edge of the absorbent article and back end edge of the absorbent core. Calculate the arithmetic mean for the L*, a* and b* values obtained from all ten measurements and report each to the nearest 0.1 unit.

Using the same prepared backsheet film samples, repeat the testing procedure on ten replicate graphics located within the portion of the backsheet between the back end edge of the absorbent core and front waist edge of the absorbent article. Calculate the arithmetic mean for the L*, a* and b* values obtained from all ten measurements and report each to the nearest 0.1 unit.

### Graphic-Free Area Test Method:

The Graphic-Free Area Test Method measures the percent area free of graphics and/or colorant on a backsheet. A flatbed scanner capable of scanning a minimum of 24 bit color at 800 dpi with manual control of color management (a suitable scanner is an Epson Perfection V750 Pro from Epson America Inc., Long Beach Calif., or equivalent) is used to acquire images. The scanner is interfaced with a computer running color calibration software capable of calibrating the scanner against a color reflection IT8 target utilizing a corresponding reference file compliant with ANSI method IT8.7/2-1993 (suitable color calibration software is Monaco EZColor or ilStudio available from X-Rite Grand Rapids, Mich., or equivalent). The color calibration software constructs an International Color Consortium (ICC) color profile for the scanner, which is used to color correct an output image using an image acquisition program that supports application of ICC profiles. The color corrected image is then segmented via color thresholding using color analysis software (a suitable image color analysis software is MATLAB R2017b available from The Mathworks, Inc., Natick, Mass.).

The samples are conditioned at about 23° C. ±2 C.° and about 50% ±2% relative humidity for 2 hours prior to testing.

The scanner is turned on 30 minutes prior to calibration and image acquisition. Deselect any automatic color correction or color management options that may be included in the scanner software. If the automatic color management cannot be disabled, the scanner is not appropriate for this application. The recommended procedures of the color calibration software are followed to create and export an ICC color profile for the scanner. The color calibration software compares an acquired IT8 target image to a corresponding reference file to create and export the ICC color profile for a scanner, which will be applied within the image analysis program to correct the color of subsequent output images.

To obtain a sample, first secure an absorbent article to a rigid flat surface in a planar configuration with the garment-facing surface up. Next, mark the on the backsheet film layer the location of the back end edge of the absorbent core, such that the portion of the backsheet between the back waist edge of the absorbent article and back end edge of the absorbent core can be distinguished from the portion of the backsheet between the back end edge of the absorbent core and front waist edge of the absorbent article. Additionally, mark the location of the landing zone region. Carefully remove the sample of backsheet film layer material from an absorbent article. A cryogenic spray (such as Cyto-Freeze, Control Company, Houston Tex.) can be used to remove the sample from the underlying and overlaying layers if necessary. If the sample contains any holes, tears, or other physical deformations, another site is to be selected. Ensure that all adhesive and nonwoven fibers have been completely removed from the sample surface.

The scanner lid is opened, and the sample carefully laid flat on the center of the scanner glass with the garment-facing surface oriented toward the glass. A scan containing the portion of the backsheet between the back waist edge of the absorbent article and back end edge of the absorbent core is acquired at 24 bit color with a resolution of 800 dpi (approximately 31.5 pixels per mm) in reflectance mode. The ICC color profile is assigned to the image producing a color corrected image. This calibrated image is saved in an uncompressed format to retain the calibrated R,G,B color values, such as a TIFF file, prior to analysis.

The calibrated image is opened in the color analysis software and cropped such that it contains only the portion or region of the backsheet selected for analysis. The image is smoothed using a 2D Gaussian filter with a sigma of 3 to blur out any individual dots of colorant. Next, utilizing a color thresholding program, a color space to perform the color thresholding is selected, for example CIELAB with its three color values L*, a*, and b*. Then a region of interest (ROI) boundary is manually drawn within a visibly discernable region of only the base color, without any graphics present, to identify its color space values. A sample with no visible base color region will be deemed to have 100% graphic coverage. The thresholding levels in all three channels of the selected color space are then manually adjusted to segment the regions of the sample that contain graphic coverage from those regions of the base color. The area of the sample containing graphic coverage is measured. The percentage of the graphic-free area of the sample is calculated by subtracting the percentage of the area of the sample containing graphics from 100%. The percentage of graphic-free area of the sample is recorded to the nearest whole percent.

In like manner, prepare, scan, and analyze the portion of the backsheet between the back waist edge of the absorbent article and back end edge of the absorbent core from six replicate backsheet samples. Calculate and report the arithmetic mean of the measured percent of graphic-free area values to the nearest whole percent.

Using the same prepared backsheet film samples, repeat the testing procedure on the portion of the backsheet between the back end edge of the absorbent core and front waist edge of the absorbent article from the six replicate backsheet samples. Calculate and report the arithmetic mean of the measured percent of graphic-free area values to the nearest whole percent.

Using the same prepared backsheet film samples, repeat the testing procedure on the landing zone region from the six replicate backsheet samples. Calculate and report the arithmetic mean of the measured percent of graphic-free area values to the nearest whole percent.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a front waist region comprising a front waist edge;
a back waist region comprising a back waist edge disposed opposite the front waist edge;
a crotch region disposed between the front and back waist regions;
a liquid permeable topsheet;
a liquid impermeable backsheet; and
an absorbent core positioned at least partially between the topsheet and the backsheet and comprising a front end edge disposed in the front waist region and a back end edge disposed in the back waist region;
wherein the backsheet comprises a plurality of graphics; and
wherein graphics of a first portion of the plurality of graphics disposed on a portion of the backsheet between the back waist edge of the absorbent article and the back end edge of the absorbent core have an opacity less than graphics of a second portion of the plurality of graphics disposed on a portion of the backsheet between the back end edge of the absorbent core and the front waist edge of the absorbent article, as measured according to the Opacity Test Method.

2. The absorbent article of claim 1, wherein the graphics of the first portion of the plurality of graphics have an opacity of between about 5% and about 90% of the opacity of the graphics of the second portion of the plurality of graphics, as measured according to the Opacity Test Method.

3. The absorbent article of claim 1, wherein a portion of the backsheet in the front waist region comprises a landing zone region, wherein an area of the landing zone region is at least 30; cm², and wherein the landing zone region has more graphic-free area than the portion of the backsheet between the back end edge of the absorbent core and the front waist edge of the absorbent article, as measured according to the Graphic-Free Area Test Method.

4. The absorbent article of claim 1, further comprising a live leg zone, wherein the live leg zone is free of graphics.

5. The absorbent article of claim 1, wherein the plurality of graphics have only one color tone.

6. The absorbent article of claim 1, wherein the backsheet is free of an underlay.

7. An absorbent article comprising:
a front waist region comprising a front waist edge;
a back waist region comprising a back waist edge disposed opposite the front waist edge;
a crotch region disposed between the front and back waist regions;
a liquid permeable topsheet;
a liquid impermeable backsheet comprising a plurality of graphics; and
an absorbent core positioned at least partially between the topsheet and the backsheet and comprising a front end edge disposed in the front waist region and a back end edge disposed in the back waist region;
wherein graphics of a first portion of the plurality of graphics disposed on a garment-facing surface of the backsheet between the back waist edge of the absorbent article and the back end edge of the absorbent core have a first opacity, as measured according to the Opacity Test Method;
wherein graphics of a second portion of the plurality of graphics disposed on the garment-facing surface of the backsheet between the first portion of the graphics and the front waist edge of the absorbent article have a second opacity, as measured according to the Opacity Test Method;
wherein the first opacity is different than the second opacity; and
wherein a portion of the backsheet between the back waist edge of the absorbent article and the back end edge of the absorbent core comprises more graphic-free area than the portion of the backsheet disposed between the back end edge of the absorbent core and the front waist edge of the absorbent article, as measured according to the Graphic-Free Area Test Method.

8. The absorbent article of claim 7, wherein the first opacity is between about 5% and about 90% of the second opacity.

9. The absorbent article of claim 7, wherein the graphics of the first portion of the plurality of graphics have an L* value of between about 10 and about 75, according to the CIE L*a*b* Color Value Test Method.

10. The absorbent article of claim 7, wherein the graphics of the second portion of the plurality of graphics have an L* value of between about 0 and about 50, according to the CIE L*a*b* Color Value Test Method.

11. The absorbent article of claim 7, wherein a portion of the backsheet in the front waist region comprises a landing zone region.

12. The absorbent article of claim 11, wherein the landing zone region is free of the plurality of graphics.

13. The absorbent article of claim 7, further comprising at least one live leg zone, wherein the live leg zone is free of the plurality of graphics.

14. The absorbent article of claim 7, wherein the portion of the backsheet disposed between the back waist edge of the absorbent article and the back end edge of the absorbent core comprises between about 20% and about 80% more graphic-free area than the portion of the backsheet disposed between the back end edge of the absorbent core and the front waist edge of the absorbent article, according to the Graphic-Free Area Test Method.

15. The absorbent article of claim 7, wherein the plurality of graphics have only one color tone.

16. The absorbent article of claim 7, wherein the backsheet is free of an underlay.

17. An absorbent article comprising:
a front waist region comprising a front waist edge;
a back waist region comprising a back waist edge disposed opposite the front waist edge;
a crotch region disposed between the front and back waist regions;
a liquid permeable topsheet;
a liquid impermeable backsheet comprising a plurality of graphics; and
an absorbent core positioned at least partially between the topsheet and the backsheet and comprising a front end edge disposed in the front waist region and a back end edge disposed in the back waist region;
wherein graphics of a first portion of the plurality of graphics disposed on a garment-facing surface of the backsheet between the back waist edge of the absorbent article and the back end edge of the absorbent core have a first opacity, as measured according to the Opacity Test Method;
wherein graphics of a second portion of the plurality of graphics disposed on the garment-facing surface of the backsheet between the first portion of the graphics and the front waist edge of the absorbent article have a second opacity, as measured according to the Opacity Test Method;
wherein the first opacity is between about 5% and about 90% of the second opacity; and
wherein a portion of the backsheet between the back waist edge of the absorbent article and the back end edge of the absorbent core comprises between about 20% and about 80% more graphic-free area than a portion of the backsheet disposed between the back end edge of the absorbent core and the front waist edge of the absorbent article, as measured according to the Graphic-Free Area Test Method.

18. The absorbent article of claim 17, wherein the graphics of the first portion of the plurality of graphics have an L* value of between about 10 and about 75, according to the CIE L*a*b*Color Value Test Method.

19. The absorbent article of claim 18, wherein the graphics of the second portion of the plurality of graphics have an L value of between about 0 and about 50, according to the CIE L*a*b* Color Value Test Method.

20. The absorbent article of claim 17, further comprising at least one live leg zone, wherein the live leg zone is free of graphics.

21. The absorbent article of claim 17, wherein the plurality of graphics have only one color tone.

22. The absorbent article of claim 17, wherein the backsheet is free of an underlay.

23. The absorbent article of claim 17, wherein the plurality of graphics are generally circular dots.

24. The absorbent article of claim 17, wherein the plurality of graphics comprise a plurality of line segment groups, wherein lines of each of the plurality of line segment groups are arranged generally parallel to one another, and wherein each plurality of line segment groups generally forms the shape of a square.

25. The absorbent article of claim 17, wherein the plurality of graphics comprise a plurality of crossed line segments.
